(19) United States
(12) Patent Application Publication
(10) Pub. No.: US 2020/0383826 A1
(43) Pub. Date: Dec. 10, 2020

(54) SYSTEM AND METHOD TO RELIEVE PAIN

(71) Applicant: Sahar Bayat, San Clemente, CA (US)

(72) Inventor: Sahar Bayat, San Clemente, CA (US)

(21) Appl. No.: 16/896,199

(22) Filed: Jun. 8, 2020

Related U.S. Application Data
(60) Provisional application No. 62/857,936, filed on Jun. 6, 2019.

(51) Int. Cl. A61F 7/02; A61H 23/02

(52) U.S. Cl. CPC A61F 7/02; A61H 23/02; A61H 2201/0207; A61H 2201/0103; A61F 2007/0078; A61H 2205/083; A61F 2007/0212; A61F 2007/0228; A61F 2007/0231; A61H 2201/5097

(57) ABSTRACT

A pain relief system and apparatus, comprising a main body formed of one or more sections adapted to surround a person's waist; a compression element, a heating element within said one or more sections operable to heat the entire area of the one or more sections of the main body and apparatus, and an adjustable belt means to maintain the main body in engagement over said person's waist. Optionally, the pain relief system may include at least one inflatable bladder adapted to create a compression force on the person's abdomen when inflated, a massage element, and may be wireless.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### CROSS REFERENCE TO RELATED APPLICATIONS

This patent application claims the benefit of U.S. Provisional Application. No. 62/857,936, filed Jun. 6, 2019, entitled METHOD AND APPARATUS TO RELIEVE PAIN.

The entire content of 62/857,936 is hereby incorporated by reference.

### BACKGROUND OF THE DISCLOSURE

### 1. Field of the Disclosure

The present disclosure relates generally to a pain relief system, especially to a menstruation pain relief device generating heat, compression pressure and massaging elements to alleviate discomfort, including abdominal, and back pain.

### 2. Description of the Related Art

Heating pads have long been used to help relieve the pain and discomfort caused by sore muscles. However, heating pads are usually an immobilizing method of applying heat to oneself as they generally require being within reach of an electrical outlet. In addition, most heating pads in the market today cannot be conveniently worn without having to hold them by hand over areas of pain to apply pressure, or heat, and relieve a given body area.

Furthermore, very few people are comfortable being seen in public using therapeutic devices, so most people do not feel comfortable using them outside their homes and in public places such as work or public events.

These are some common problems that women face when suffering from painful menstrual cramps. These cramps are caused by the blood supply to the uterus being constricted while the muscles in the lower abdominal area contract. The application of heat and pressure to this area is often used to treat this pain because it increases blood-flow and reduces muscle stiffness, thus effectively soothing the pain without any need for medication. Moreover, menstruation also causes back pain, which this apparatus soothes in a similar fashion. In addition, a vast number of women will miss work, school or important functions because they don't have a suitable portable option to relieve the pain.

Over the years, many attempts have been made without success, to address these issues. For example, U.S. Pat. No. 5,928,275 to Yates (1999) demonstrates the design for a belt-like body heating apparatus. The belt employs portable methods of heating that focuses primarily on the distribution of said heat throughout the entire body by means of warming chemicals (one time use liquid/gel packets that heat up for a certain amount of time) being placed in pouches on the belt. This is both wasteful and harmful to the environment and the packets must be replaced every time the device is used.

As another example, U.S. Pat. No. 8,191,550 to Lee (2012) discloses an apparatus to relieve menstrual cramping which includes one or more pads having an inner and outer side, each outer side being semi-rigid and each inner side being flexible. The outer side of each pad is connected to one or more straps having first and second ends. A fastener is attached to the first end of each strap and a corresponding second fastener attaches to the second end of each strap. The apparatus includes a variable compression drive located proximate to one pad to create a compression force through each strap when the first and corresponding second fasteners connect. The drive includes an outer drum shell, an inner rotator having an inner diameter that is treaded, a motor which communications with the inner rotator, and a threaded shaft which engages the inner diameter of the inner rotator. A tab attaches the threaded shaft to the strap. This device is bulky, cannot be concealed under regular garment, and does not provide any pressure or massaging comfort to the person in pain. In addition, only portions of the device are heated making this difficult to use when a user suffers from pain which is not localized in one small area.

U.S. Pat. No. 4,607,624 to Jefferson, et al. (1986) is a further example, and discloses a heating pad massager that is a massaging and heating pad combination for application to the body in which both massaging vibration intensity and temperature may be controlled, primarily intended for use in the relief of the pain and discomfort of cramps and muscle tension and other suffering attendant to the menstrual cycle. However, this device requires the user to keep the device plugged into an electrical outlet at all times and does not allow the user to heat the entire surface of the heating pad.

U.S. Pat. No. 5,701,608 to Kohn (1997) discloses an undergarment apparatus and method for reducing menstrual cramping. The undergarment apparatus and method for reducing menstrual cramping includes an elastic panel mounted to at least one of a front portion and a rear portion of an underpant brief in a manner exerting inward pressure across a lower torso region of an individual wearer. At least one movable pressure bearing insert is formed for positioning between the elastic panel and the lower torso region at a plurality of positions along the elastic panel. The elastic panel cooperates with an apex portion or small area side of the pressure bearing insert to exert substantial localized point pressure on a relatively small area of the torso region for mitigation of menstrual cramping. However, this device requires the user to manually move the pressure inserts and lacks any sort of heating element.

Similarly, U.S. Pat. App. No. US2011004839A1 to Lee (2011), discloses a compression undergarment for relief of menstrual pain and a related method. As with the '624 patent, this device requires the user to manually change compression pads and does not provide heat for the user.

As can clearly be ascertained from the above examples, the solutions so far have not been satisfactory for users. However, the present disclosure aims to solve the above problem thanks to a portable, wireless and discreet heating pad which can be worn under clothing, thereby overcoming the limitations imposed by the prior art.

### SUMMARY OF THE DISCLOSURE

In one design of the present disclosure, it is embodied as a pain relief system which can be discreetly worn in public and under garments. The pain relief system can be heated throughout its entire surface and can be worn around a person's abdominal region, particularly the waist. Optionally, the surface area of the main body can be divided into sections, which can also be heated independently of each other.

In addition, the pain relief system is equipped with controllable heating elements throughout the entire system allowing the user to apply heat to the entire circumference of their waist if they desire, or just specific sections. Thus, thanks to a controller, a user can choose which section(s) to heat and control heat settings for maximum pain relief and comfort.

In some embodiments, the heating elements are enclosed with, or my be integral to, compression pads to create compression and gentle force on the areas of pain and to move the heating elements closer to the same. Preferably, the compression pads are made from open cell polyurethane foam.

The system also includes an adjustment means operable to allow a person to tighten or loosen the main body to fit their body comfortably.

The system can also tapered at the bottom allowing a user to heat lower parts of the abdomen, lower back, or both, which tend to be particularly sore for women during menstruation. In one possible embodiment, the lower portion of the device extends lower on a person's back to address back soreness as well. In other embodiments, a flap extends below the main body of the system to cover the user's lower abdomen and sits between the users hips so that the user may sit without the flap folding up.

To further relieve pain, the system also includes optional massaging elements and inflatable bladders that can be filled with air to create compression and a gentle force on the areas of pain. Inflating the inflatable bladders also brings the heating elements closer to the sore areas.

In addition, the system can be wireless and rechargeable using one or more battery packs. This way, a user does not need to stay close to a wall plug in order to apply heat to their pain areas.

In another possible embodiment the pain relief system comprises a main body formed of one or more sections adapted to surround a person's waist, at least one compression element adapted to create a compression force on the person's abdomen, a heating element within the one or more sections, wherein the heating element is operable to heat the entire area of the one or more sections of the main body, and an adjustable belt means to maintain the system in engagement over the person's waist.

In another possible embodiment, the heating element within the section is adapted to heat the section independently of or in conjunction with the other sections.

In another possible embodiment, wherein the belt means comprises at least one of a group consisting of a hook and attachment, Velcro, magnets, a zipper, snaps, a manual belt, or a hook and handle combination.

In another possible embodiment, at least one massaging element is adapted to massage a given body area.

In another possible embodiment, a tapered bottom cutout is arranged corresponding to a person's lower abdomen area.

In another possible embodiment, the system is formed of a suitable fabric material and wherein the suitable fabric material is thin and adapted to stretch.

In another possible embodiment, the system is covered with a detachable fabric material shell.

In another possible embodiment, the system is wirelessly connected to a power supply.

In another possible embodiment, the system is rechargeable.

In another possible embodiment, the system further includes a controller assembly comprising a controller attached to the system adapted to control heat, compression, and massage features of the system.

In another possible embodiment, the controller is operable to accept user commands via a remote-control interface.

In another possible embodiment, the system further comprises a flap extending below the main body, wherein the flap is adapted to cover a person's lower abdomen area.

In another possible embodiment, the belt means comprises at least one of a group consisting of a hook and attachment, Velcro, magnets, a zipper, snaps, a manual belt, or a hook and handle combination.

In another possible embodiment, the detachable fabric material shell is attached to the system using at least one of a group consisting of snapping elements, magnetic elements, a zipper, or Velcro.

In another possible embodiments, the compression element is comprised of at least one of a group consisting of polyurethane foam, an inflatable bladder adapted to create a compression force on the person's abdomen when inflated, or fabric.

In another possible embodiment the pain relief system comprises a main body formed of one or more sections adapted to surround a person's waist, at least one inflatable bladder adapted to create a compression force on the person's abdomen when inflated, a heating element within said one or more sections, wherein said heating element is operable to heat the entire area of the one or more sections of said main body, and an adjustable belt means to maintain the system in engagement over said person's waist.

As will be obvious to anyone skilled in the art, numerous changes and modifications can be made to the design of this disclosure without deviating from the scope of the present disclosure.

### BRIEF DESCRIPTION OF THE DRAWINGS

FIG. 1 is a front view of one embodiment of the present disclosure.

FIG. 2 is a front view of another embodiment of the present disclosure showing an alternative embodiment of adjustment means.

FIG. 3 is rear view of another embodiment of the present disclosure showing internal elements and a detachable power adapter.

FIG. 4 is a view of one embodiment of a remote control adapted to control various settings of the present disclosure.

FIG. 5 shows one embodiment of the present disclosure in environmental settings and how it can be positioned on a user's body.

FIG. 6 is a rear view of another possible embodiment of the present disclosure.

FIG. 7 is a front view of another possible embodiment of the present disclosure.

FIG. 8 shows another embodiment of the present disclosure with inner elements such as heating elements.

FIG. 9 shows a cross sectional view of another embodiment of the present disclosure showing multiple sections, heating elements, massaging elements and compression elements.

FIG. 10 shows another embodiment of the present disclosure with inner elements such as heating elements on selected sections.

FIG. 11 shows a cross sectional view of another embodiment of the present disclosure showing multiple sections, heating elements, massaging elements and compression elements on selected sections.

FIG. 12 shows another embodiment of the present disclosure with inner elements such as heating elements on selected sections.

FIG. 13 shows a cross sectional view of another embodiment of the present disclosure showing multiple sections, heating elements and compression elements on selected sections.

The same elements or parts throughout the figures of the drawings are designated by the same reference characters, while equivalent elements bear a prime designation.

### DETAILED DESCRIPTION OF THE DISCLOSURE

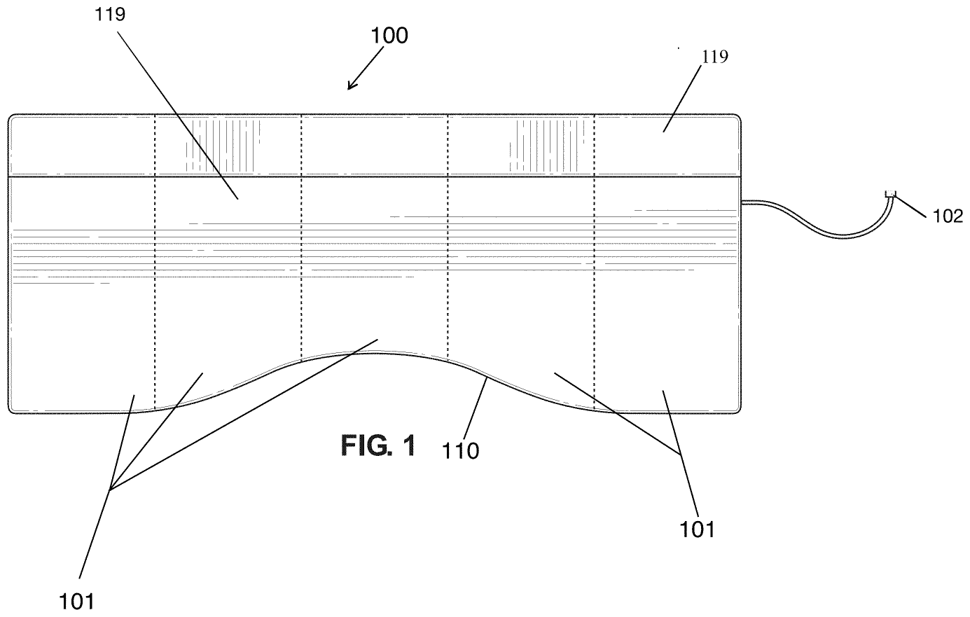

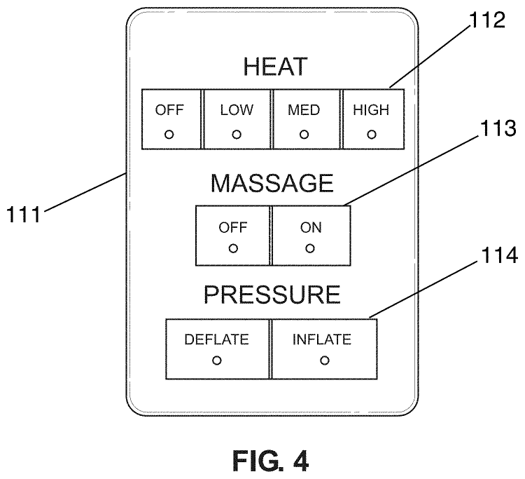

Referring now to the drawings and the characters of reference marked thereon, FIG. 1 shows one embodiment of the pain relief system 100. The system shows a main body 119 and optional sections 101. In one embodiment, the pain relief system has two or more sections 101. Each section of the system includes heating elements throughout the system, such that the entire system and its entire surface area can be heated. In this embodiment, each section 101 operates independently from the other section. In another embodiment, these sections can be controlled by a user using a remote control 111 as shown in FIG. 4. In another embodiment, a user can turn on the heating elements in all sections or turn them all off at once. In yet another embodiment, the system is not divided into sections and the entire surface area of the system is adapted to heat to various levels.

The system operates wirelessly, and without having to be connected to a wall plug for power. In one wireless embodiment, as shown in FIG. 1, the system is rechargeable, and a user can plug the system in for charging thanks to a rechargeable adapter 102. This way, a user can move about while using the system without having to be near an electrical outlet.

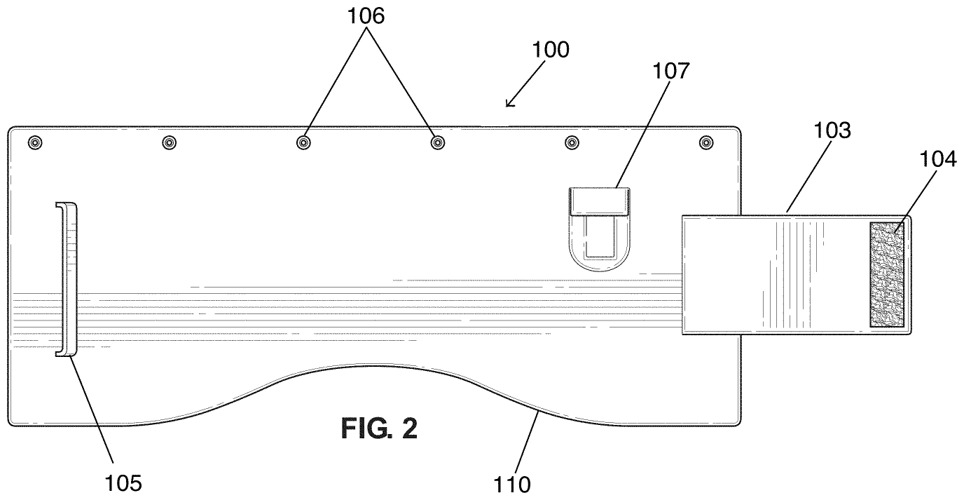

FIG. 2 shows another embodiment of the system. The figure shows the outside of the system, or the side that would be facing out when a user is wearing the system. An adjustable belt means 103 is used to maintain the system in engagement with a person's waist. In the illustrated embodiment, the adjustable belt means is a Velcro pad 104 supported by a loop 105. In another possible embodiment, the adjustable belt means are a hook and an attachment adapted to connect to that hook and allow a user to tighten or loosen the system around a person's lower abdomen. In other embodiments, the device can be secured on a person's body thanks to other coupling means on the device such as Velcro, magnets, a zipper, snaps, a manual belt, a hook and handle combination or other coupling means.

In these and other possible embodiments, the system is formed of a suitable fabric material which is thin and flexible allowing the system to be worn under garment and discreetly in public.

In another embodiment, such as the one shown in FIG. 2, the system also has a detachable cover so that a user can remove the outer cover and wash it to keep the system clean. The system shown in FIG. 2 includes attachments 106 where the cover can be attached to the system or removed for washing. While the illustrated embodiment shows snapping elements, the attachments can be magnetic, a zipper, Velcro or any other mechanical attachment.

FIG. 2 also shows an optional pocket 107 where a user can store the remote control. In another embodiment, the controllers are attached to the system allowing the user to control the system right from the system itself.

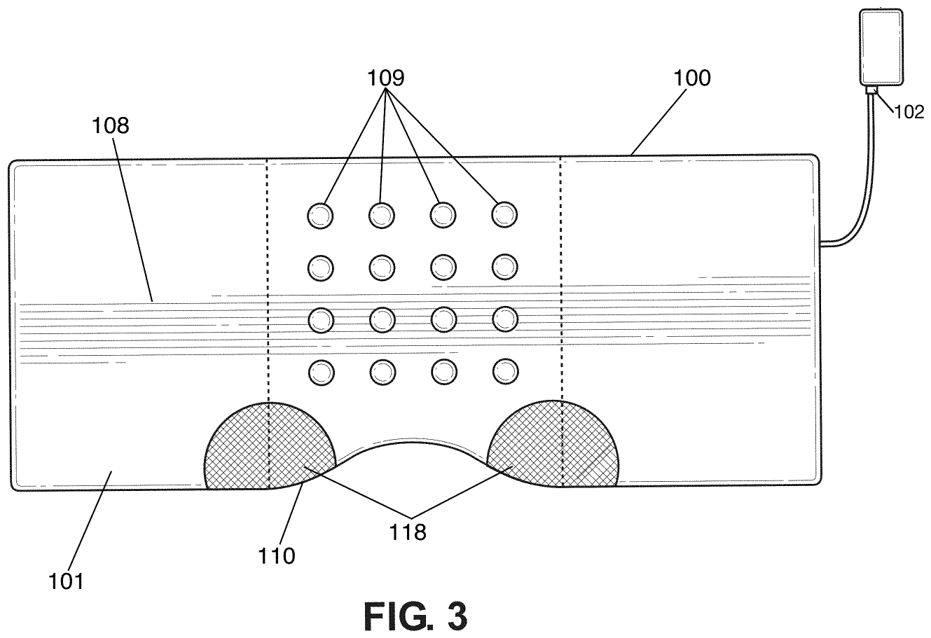

FIG. 3 shows another embodiment of the present disclosure with internal elements illustrated. In this embodiment, in addition to the independent sections 101 and heating elements 108 throughout the system, the pain relief device further includes massaging elements 109. The massaging elements can be located in specific sections 101 or throughout the entire system. In another embodiment, the massaging elements within a section 101 can be controlled independently by a user. The massaging elements are optional and other embodiments do not include massaging elements.

In the illustrated embodiment of FIG. 3, the system has at least one inflatable bladder 118. The bladder can be controlled by a user and filled with air to inflate or deflate the bladder. This creates a gentle compression force on the person's body as the bladder is inflated and may be controlled by the user. In one embodiment, the inflatable bladder is sandwiched within a section, and has heating elements on the surface such that a user can apply gentle force on the body and push the heat closer to the pain areas to increase pain relief.

In one embodiment, the inflatable bladder 118 can be inflated or deflated manually, whereas in other embodiments, the bladder can be controlled electrically thanks to a controller, or a combination of both manual and electrical inflation and deflation can be used, which allows the user to choose the method of inflation or deflation, and the level of inflation of the bladder, thereby controlling the compression applied.

FIGS. 1 through 3 also show possible embodiments of the system that include a tapered bottom cutout 110. The tapering of the extension lobe 110 is arranged corresponding to a person's lower abdomen, allowing women to apply heat to certain body areas that tend to be sore and painful during menstruation. The tapered section 110 can be in the front, or both the front and the back, thereby helping deliver heat, massaging, compression force, or any combination thereof to the lower abdomen and lower back, which tend to be sore during menstruation.

FIG. 4 shows one embodiment of a remote control 111 to help users control various settings such as heat settings 112, massage settings 113, and pressure setting 114. The remote can be detached from the system 100 or can be coupled to the system 100 itself.

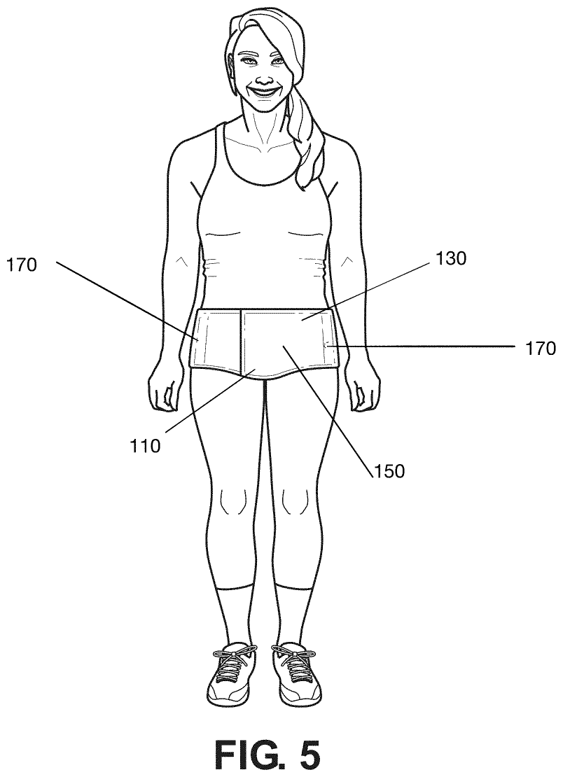

In one embodiment, a user can wrap the pain relief system 100 around their waist and lower abdomen as shown in FIG. 5. The user can use the belt adjustment means to keep the system positioned closely to the pain areas.

In some embodiments, the system includes an automatic shut-off option which can be pre-programed or set by a user. In one specific embodiment, the auto shut off option can be set to 1 hour, 2 hours or 3 hours, or incrementally by 30 minutes intervals.

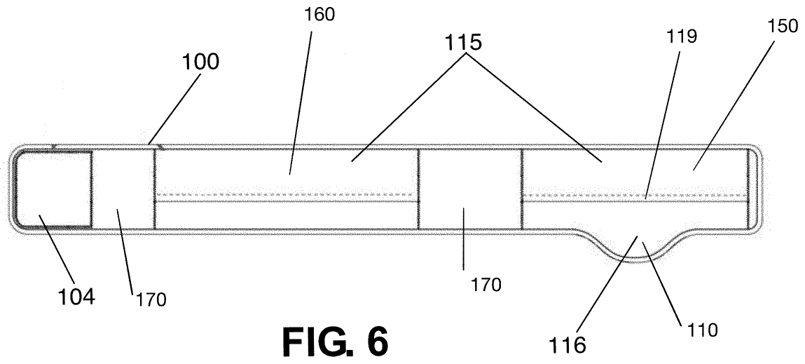

FIG. 6 shows another possible embodiment of the system with heating element pockets 115 with an opening in the middle of the heating element pockets 115 operable to hold a removable heating element. Optionally, the upper cover of the pocket may overlap the lower cover of the pocket to further contain the heating element while the user is wearing the system 100.

In this and other embodiments, a front section flap116 extending below the main body 119 of the pain relief system 100 to cover the sections of the user's abdomen where the user's ovaries may be located can be added or used to replace the tapered bottom cutout. In some embodiments, the front section flap 116 may be narrow enough to sit between the user's hips to reduce the risk of the front section flap116 folding up when the user sits because it will not be sitting at the point where the user's hip joint bends.

Also illustrated in FIG. 6, the Velcro pad 104 may come in various shapes and sizes to accommodate user with different waist sizes.

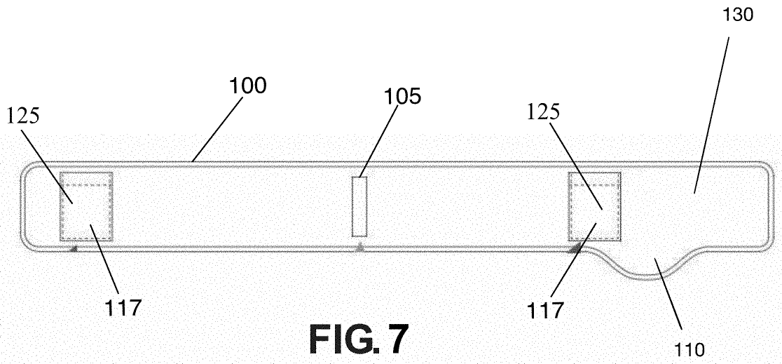

In these and other embodiments, multiple battery pockets 117 may be used to house multiple batteries, as illustrated in FIG. 7 which shows another possible embodiment of the system 100. Using multiple batteries will allow the system to operate for longer periods of time between recharges, thereby prolonging the treatment of the user's symptoms without the need to charge the batteries or connect the system 100 to an electrical outlet.

In one embodiment, the system has a detachable fabric material shell which is attached to the system using at least one of a group consisting of snapping elements, magnetic elements, a zipper, or Velcro. In another embodiment, the system and system are enveloped in detachably removable cover which can be reused and washed separately before reuse.

In another embodiment, the system and system comprise a flap extending below the main body, where the flap is adapted to cover a person's lower abdomen area.

In an alternative embodiment, the present disclosure is embodied as a pain relief system and system which includes a main stretchable body formed of one or more sections adapted to surround a person's waist, an inner portion to be placed against a person's body and an outer portion facing outwardly. The system and system may further include an extension lobe arranged corresponding to a person's lower abdomen area and adapted to be placed against the person's lower abdomen area. The system and system further include at least one heating element within one or more sections, where the heating element is operable to heat the entire area of the one or more sections of said system.

In some embodiments, the heating element is operable to heat the entire area of the one or more sections of the system. In other embodiments, the heating elements is one or more wired heating elements disposed as an S shaped element to cover the entire surface area of one or multiple sections of the heating and pain relief system. In other embodiments the heating elements extend throughout a substantial surface area of the system stopping shortly at the perimeter of the pain relief system and do not cover the perimeter or any stitching at the top and bottom of the pain relief system.

In another embodiment, the compression elements are couplably removable to the system and can be inserted in pockets within the system, be integrally and structurally connected to the system or be removed if a user wants to remove them. In one particular embodiment, the compression elements are a compression foam, an inflatable bladder, a compression pad, a therapeutic compress, and/or a polyurethane foam pad adapted to create a compression force on the person's abdomen.

In one particular embodiment, the compression element is disposed between the outer portion and the heating element to exert a relieving pressure on the person's waist and abdomen and adapted to render said heating elements closer to a person's body to relieve pain. Additional adjustable means are structurally coupled to the system to maintain the system in engagement over said person's waist.

In one embodiment, the system and apparatus include massaging elements, heating elements and compressions elements which can be operable independently of or in conjunction with one another. In this embodiment, the user can either inflate a bladder for compression or deflate the bladder, or remove the compression pad manually to control the compression element. The heating and massaging elements and compression elements in non-manual embodiments can be controlled via a controller by the user.

In another embodiment, the system and apparatus also include a battery electrically coupled to the heating element rendering the apparatus rechargeable. In some embodiments, the user can connect the device to an external power outlet for charging while in other embodiments, the system and apparatus are wirelessly rechargeable.

The present disclosure can also be embodied as a method for relieving menstrual cramping pain, the method comprising the steps of: positioning a pain relief apparatus 100 on a person's waist; arranging an extension lobe 110 of the pain relief apparatus of a person's lower abdomen; tightening said pain relief apparatus on a person's waist using the apparatus's adjustable means 103; powering said apparatus to apply heat on said person's body (with controller assembly 111 for example and a remote control 111).

In another embodiment, the method also includes the steps of inflating an inflatable bladder with air 140, 118. (See. FIG. 11). In yet another embodiment, the method also includes the steps of powering at least one of a heating element 109, a compression element 140 and a massaging element 109 independently or in conjunction with one another at a user's discretion.

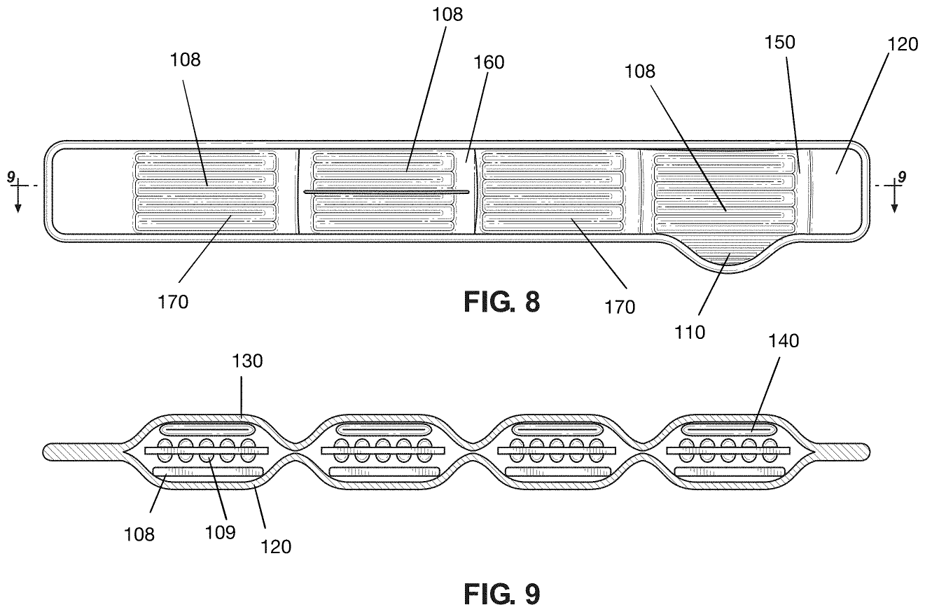

In one embodiment, the present disclosure is embodied as a pain relief system 100 including a pain relief device with a main body 119 formed of one or more sections 101. The main body 119 is adapted to surround a person's waist as shown in FIG. 5. The system 100 also has an inner portion 120 to be placed on the person's waist and an outer portion 130 facing outwardly and away from the person. In this embodiment, the system and device also comprise at least one compression element 140 which is adapted to create a compression force on a person's body. The heating elements 108 can be within one or more sections 101 and heating element is operable to heat a substantial area as shown in FIGS. 8, 10 and 12 and of the one or more sections of said apparatus.

In most embodiments, whether the system and the main body are split into multiple sections 101 or at least one main section covering the entire body, it is one objective of the present disclosure that the heating elements cover the entire section 101. In some embodiments, the heating elements stop short of the perimeter and edge, in other embodiments, the heating elements cover the center and most of the surface area of the heated section.

In one embodiment, the heating element is a wired heating element 108 twisted in an S-shape to cover the majority of a section as shown in FIGS. 8, 10 and 12. In other embodiments, the heating elements are akin the elements 109 shown in FIG. 3 and cover an entire section.

In one embodiment, the system includes two sections, one covering the front of a person and abdomen: this section is heated throughout and includes compression elements whereas the second section does not include any heating elements or compression elements.

In another embodiment as shown in FIG. 8, the system includes a plurality of sections 101, one to cover a person's abdomen, a front portion 150, one to cover a person's back, a back portion 160 and two side portions 170 to cover a person's side. In one particular embodiments, all sections 101 and portions 150, 160 and 170 include heating elements 108 throughout, compression elements 140 in all sections and massaging elements in all sections as shown in the cross sectional FIG. 9.

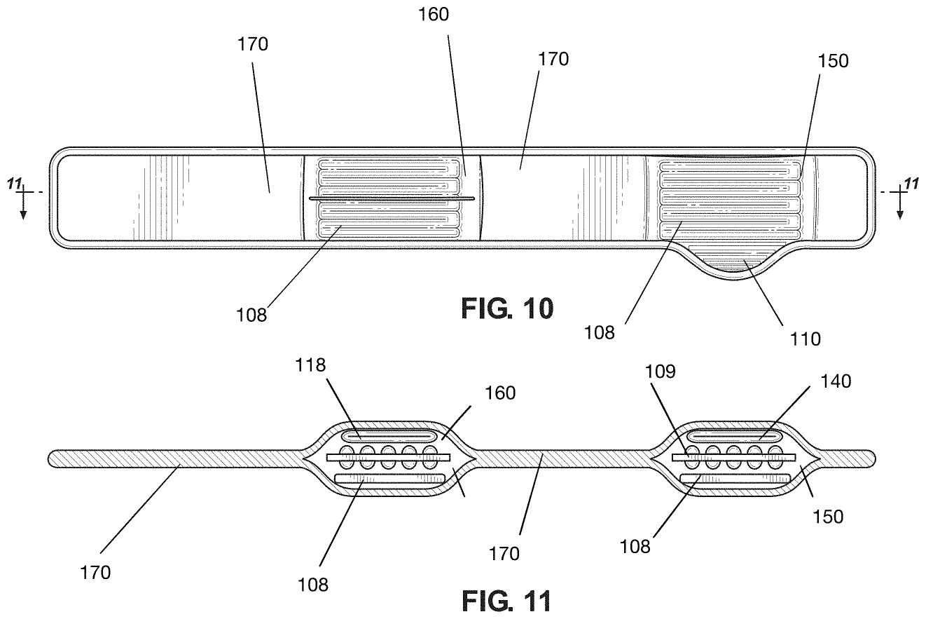

In another embodiment as shown in FIGS. 10 and 11, the system includes two main sections 101, one to cover a person's abdomen, a front portion 150, one to cover a person's back, a back portion 160. The sides of a person do not include any heating elements, compression elements or massaging elements and are just stretchable fabric. In one particular embodiments, all sections 101 and portions 150, 160 include heating elements 108 throughout, compression elements 140 in all sections and massaging elements in all sections as shown in the cross-sectional FIG. 11.

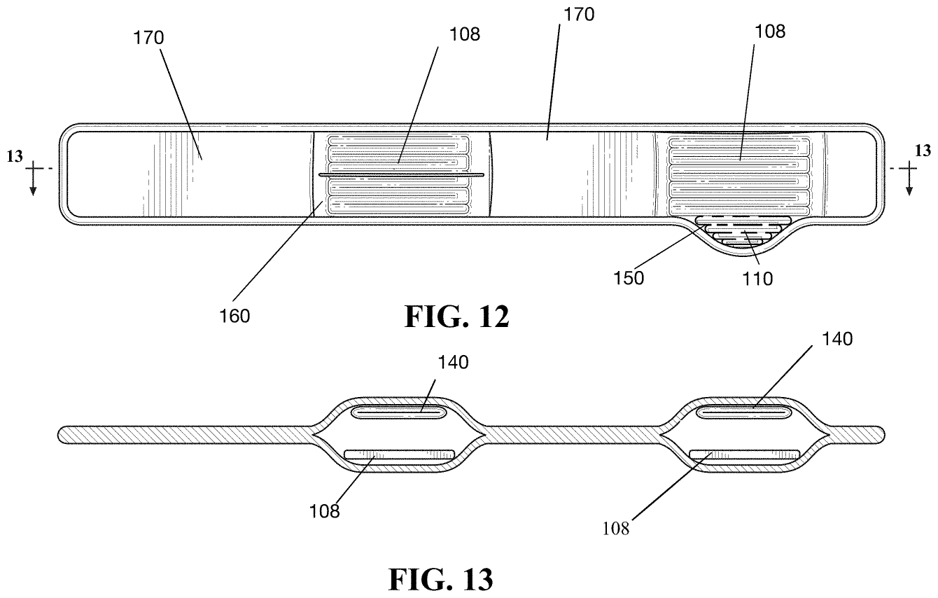

In yet another embodiment as shown in FIGS. 12 and 13, the system includes two main sections 101, one to cover a person's abdomen, a front portion 150, one to cover a person's back, a back portion 160. The sides of a person do not include any heating elements, compression elements or massaging elements and are just stretchable fabric. In one particular embodiments, all sections 101 and portions 150, 160 include heating elements 108 throughout and compression elements 140 in all sections but do not include massaging elements as shown in the cross-sectional FIG. 13.

In one embodiment, the system also includes adjustable means 103 to maintain the system 100 and main body 119 in engagement over the person's waist; and when the compression element is placed between the outer portion 130 and the heating element 108, the compression element renders the heating element closer to the person's waist and applies a relieving compression force on a person's body.

In another embodiment, the system's compression elements 140 can be a compression foam, an inflatable bladder, a pressure pad, a therapeutic compress, a polyurethane foam and/or a compressible material or a combination and equivalent thereof adapted to create a compression force on the person's body.

In one particular embodiment, the compression element 140 is removably couplable to the main body. That is, the compression elements can be inserted into the main body or removed according to user's discretion. In one particular embodiment, the compression element can be inserted in pockets and in section pockets as shown in FIG. 6. Here, FIG. 6 shows a pocket 115 with opening and closure 117 in the middle of the main body. This embodiment ensures the compression element from sliding down. In other embodiment, the opening/closure 117 of the pocket or pockets 115 can be located either on the side of the section and opening, or at the bottom of the main body.

In one embodiment, the heating element or heating elements 108 within a section can heat one or more sections independently of another or can all heat up all sections in conjunction with one another. For example, FIGS. 6, 10 and 11 show an embodiment where only the front of the body and back are heated and compressed and include heating elements and compression elements whereas FIG. 8 shows an embodiment where the entire system is heated with heating elements throughout the main body. In an embodiment as shown in FIG. 8 a user can select thanks to a remote control 111 and a controller assembly 111 which sections to engage with heating, pressure or massage.

In another embodiment, the adjustable means can be a hook and loop attachment, Velcro®, one or more magnets, a zipper, snaps, a manual belt, and/or a hook and handle combination thereof which would allow a user to tighten and secure said system on the person's body. In one embodiment, the main body is made out of stretchable material such that a user can tighten the system to their liking, an use the adjustable means to make sure that the system stays in place. FIG. 2 for example shows a loop 105 and hook and loop or Velcro® patch on 104. FIG. 6 shows a different embodiment of how the adjustment means can be used with the system.

In one particular embodiment, the system 100 includes a an extension lobe 110 arranged corresponding to a person's lower abdomen area where the system 100 is adapted to heat a person's lower abdomen and adapted to exert pressure on said lower abdomen area to relieve a lower abdomen pain, especially menstrual cramp pain.

In another embodiment, the system 100 also comprises one or more massaging element 109 adapted to massage a given body area and where the massaging element are disposed within the one or more sections 101 of the main body 119 as shown in FIGS. 3, 9 and 11. In one embodiment the massaging elements are only on the back portion 160 of the system 100 to massage the lower and mid back of a person. In other embodiments, the massaging elements can be placed on the side portions 170 and/or on the front portion 150. In some embodiments, the massaging elements 109 are disposed between the compression elements 140 and the inner portion 120, in other embodiments, the massaging elements and heating element are combined in one heating and massaging element. In yet another embodiment, the massaging elements 109 are between the compression element 140 and the heating element 108, whereas in an alternative embodiment, the massaging elements 109 are between the heating elements 108 and the inner portion 120 of the main body 119.

In one embodiment, the system 100 is formed of a thin and stretchable fabric material such that a user can stretch the system and the main body of the system on the person's waist and body and where the system is concealable under a person's clothing as shown in FIGS. 6, 7, 8, 10, 12.

In another embodiment, the system 100 and main body 119 is enveloped in a detachable shell adapted to be washed and reused. In one particular embodiment, as shown in FIG. 2 shows, the shell can be attachable at the top of the main body as shown in 106 thanks to cover/shell attachments.

In another embodiment, the system 100 also includes at least one battery 125 electrically coupled to the heating element. In embodiments which include inflatable bladder as compression elements and/or massaging elements, the compression elements and massaging elements are also connected to the battery or batteries 125 for power.

In one particular embodiment, a single battery 125 can provide enough power to power one or more heating elements, compression elements, and/or massaging elements. In another embodiment, the battery 125 can be received in battery pockets 117 and stored in those pockets disposed on outer portion of the system 130.

In another embodiment, all the heated elements from all sections are connected to the one battery. In an alternative embodiment, where the system 100 only includes two main sections 101, one in the front portion of the system 150 to cover the front and abdomen of the person and one in the back portion 160 to cover the backside of a person, the embodiment includes two batteries where one operate the heating elements of the front portion and another battery to operate the heating elements of the back portion.

In an alternative embodiment where 2 batteries operate the front and back portion and heating elements of the front and back portions, the batteries are set in pockets 117 disposed on the side portions of the main body.

In another embodiment, the system 100 is rechargeable. Here, the battery or batteries can be taken out and charged or the batteries are on the main body and a user can connect the System 100 to an external power source thanks to USB connections, a plug or other electrical connections through a rechargeable adapter 102 as shown for illustrative purposes in FIG. 1. In yet an alternative embodiment, the system can be wirelessly connected to an external power supply.

In one embodiment, the user can charge the batteries by removing them from the belt and recharging them, plugging them to an external power source via USB or other for recharging or while the belt is on user can plug the system/system to a power source such as computer/car charger etc.

In one embodiment, the system 100 also incudes a controller assembly 111 including a controller 111 removably attached to the main body adapted to control a heat, a compression, and a massage feature of the system 100. In a particular embodiment as shown in FIG. 4, the system 100 comes with an external pocket 107 where a user can stow the remote control 111.

In one embodiment, the present disclosure is embodied in a pain relief system 100 which includes a main stretchable body 119 formed of one or more sections 101 adapted to surround a person's waist, an inner portion 120 to be placed against a person's body and an outer portion 130 facing outwardly. The system 100 also includes an extension lobe 110 arranged corresponding to a person's lower abdomen area and adapted to be placed against the person's lower abdomen area. The system also includes at least one heating element 108 within the one or more sections, wherein said heating element is operable to heat the entire area of the one or more sections. Here, where the embodiment includes an extension lobe 110, the heating elements and/or compression elements can extend all the way to the bottom of the extension lobe to provide heat, pressure and pain relief to a person's lower abdomen and to cover a woman's ovaries and relief menstrual cramps and pain. The system 100 also includes at least one couplably removable compression element 140 disposed between said outer portion 130 and said heating element 108 to exert a relieving pressure on the person's waist and abdomen and adapted to render the heating elements 108 closer to a person's body to relieve pain. The system also includes adjustable means 103 to maintain the system 100 and the main body 119 in engagement over the person's waist.

In another embodiment, the removably compression element 140 is either a compression foam, an inflatable bladder, a compression pad, a therapeutic compress, and a polyurethane foam pad or a combination thereof adapted to create a compression force on the person's body.

In another embodiment, the system also includes massaging elements 109; and the heating elements 108, the massaging elements 109 and the compression elements 140 are operable independently of one another or in conjunction with one another as shown in FIGS. 9, 11 and 13.

In an alternative embodiment, the system 100 includes one or more batteries 125 electrically coupled to the heating element where the system is rechargeable.

In one embodiment where the system 100 also includes a shell or detachable fabric material shell, the shell/detachable fabric can be attached to the main body 119 using snapping elements, magnetic elements, a zipper, and/or Velcro® or a combination thereof.

In another embodiment the system 100 also includes a flap or extension lobe 110 extending below the main body 119, such that the extension lobe is adapted to cover a person's lower abdomen area. In some embodiments, the extension lobe also includes heating elements extending to the edge of the extension lobe as shown in FIGS. 8, 10 and 12. In some embodiments, the extension lobe also includes compression elements extending to the edge of the extension lobe 110 to create a compression force on a person's body, and a compression force on front of a person and over the ovaries region often responsible for menstrual cramping.

In one embodiment, the heating element within the one or more sections, where the heating element is operable to heat a substantial area of the one or more sections of the system. Here the substantial area is defined as the majority of a given section, and the majority of its surface areas.

In a particular embodiment, the system also includes a remote controller 111 and the system is operable to accept user commands via remote-control interface.

Additional advantages and modifications will readily occur to those skilled in the art. Therefore, the disclosure in its broader aspects is not limited to the specific details, and representative devices shown and described herein. Accordingly, various modifications may be made without departing from the spirit or scope of the general inventive concept as defined by the appended claims and their equivalents.

1. A pain relief system comprising:
a) a main body formed of one or more sections adapted to surround a person's waist, an inner portion to be placed on said person's waist and an outer portion facing outwardly;
b) at least one compression element adapted to create a compression force on a person's body;
c) a heating element within said one or more sections, wherein said heating element is operable to heat a substantial area of the one or more sections of said main body; and
d) an adjustable means to maintain said main body in engagement over said person's waist; and wherein said at least one compression element is placed between said outer portion and said heating element such that the at least one compression element renders said heating element closer to said person's waist and applies a relieving compression force on a person's body.

2. The pain relief system of claim 1, wherein said compression element is at least one of a compression foam, an inflatable bladder, a pressure pad, a therapeutic compress, a polyurethane foam and a compressible material adapted to create a compression force on a person's abdomen.

3. The pain relief system of claim 1, wherein said compression element is removably couplable to said system.

4. The pain relief system of claim 1, wherein said heating element within said section is adapted to heat said section independently of or in conjunction with other sections.

5. The pain relief system of claim 1, wherein said adjustable means comprise at least one of a hook and loop attachment, Velcro, one or more magnets, a zipper, snaps, a manual belt, or a hook and handle combination allowing a user to tighten and secure said system on the person's body.

6. The pain relief system of claim 1 further comprising an extension lobe arranged corresponding to a person's lower abdomen area wherein said system is adapted to heat a person's lower abdomen and adapted to exert pressure on said lower abdomen area to relieve a lower abdomen pain.

7. The pain relief system of claim 1 further comprising at least one massaging element adapted to massage a given body area and wherein said massaging element is disposed within said one or more sections of said main body.

8. The pain relief system of claim 1, wherein said system is formed of a thin and stretchable fabric material such that a user can stretch said system on the person's waist and wherein said system is concealable under a person's clothing.

9. The pain relief system of claim 8, wherein said system is enveloped in a detachable shell adapted to be washed and reused.

10. The pain relief system of claim 1, wherein said system further comprises a battery electrically coupled to said heating element.

11. The pain relief system of claim 10, wherein said system is rechargeable.

12. The pain relief system of claim 10, wherein said system is wirelessly connected to an external power supply.

13. The pain relief system of claim 10 further comprising a controller assembly including a controller removably attached to said main body adapted to control a heat, a compression, and a massage feature of said system.

14. A pain relief system comprising:
a) a main stretchable body formed of one or more sections adapted to surround a person's waist;
b) an inner portion to be placed against a person's body and an outer portion facing outwardly;
c) an extension lobe arranged corresponding to a person's lower abdomen area and adapted to be placed against the person's lower abdomen area;
d) at least one heating element within said one or more sections, wherein said heating element is operable to heat an entire area of the one or more sections;
e) at least one couplably removable compression element disposed between said outer portion and said heating element to exert a relieving pressure on the person's waist and abdomen and adapted to render said heating elements closer to a person's body to relieve pain; and
f) an adjustable means to maintain said main body in engagement over said person's waist.

15. The pain relief system of claim 14, wherein said removably compression element is at least one of a compression foam, an inflatable bladder, a compression pad, a therapeutic compress, and a polyurethane foam pad adapted to create a compression force on the person's body.

16. The pain relief system of claim 14 further comprising massaging elements; and wherein said heating elements, said massaging elements and said compression elements are operable independently of or in conjunction with one another.

17. The pain relief system of claim 14 further comprising at least one battery electrically coupled to said heating element; wherein said system is rechargeable.

18. A method for relieving menstrual cramping pain, the method comprising steps of:
a) positioning a pain relief apparatus on a person's waist;
b) arranging an extension lobe of the pain relief apparatus of a person's lower abdomen;
c) tightening said pain relief apparatus on a person's waist using an apparatus's adjustable means; and
c) powering said apparatus to apply heat on a person's body.

19. The method of claim 18 further comprising the steps of inflating an inflatable bladder with air.

20. The method of claim 18 further comprising the steps of powering at least one of a heating element, a compression element and a massaging element independently or in conjunction with one another at a user's discretion.
